(19) United States
(12) Patent Application Publication
(10) Pub. No.: US 2007/0050875 A1
(43) Pub. Date: Mar. 8, 2007

(54) Face shield for helmet and method for making same

(76) Inventors: Kirby Bentley, Stamping Ground, KY (US); Karen Lynn Bentley, Stamping Ground, KY (US)

Correspondence Address: David O. Simmons;Galasso & Associates LP, P.O. Box 26503, Austin, TX, 78755-0503, US

(21) Appl. No.: 11/210,174

(22) Filed: Aug. 22, 2005

(51) Int. Cl. A61F 9/00

(52) U.S. Cl. 2/15

(57) ABSTRACT

A face shield for a helmet comprises a face shield body including end portions and a central portion extending between the end portions. Each one of the end portions is configured for enabling attachment of the face shield to a respective attachment portion of a helmet. The central portion is made from transparent polymeric material. The polymeric material in a first area of the central portion exhibits non-photochromic properties at least one layer of the polymeric material in a second area of the central portion exhibits photochromic properties.

### FIELD OF THE DISCLOSURE

The disclosures made herein relate generally to motorcycle and race vehicle helmets and, more particularly, to face shields for motorcycle and race vehicle helmets.

### BACKGROUND

Regardless of whether a helmet is used in a motorcycle application or automobile application, the need arises to protect a wearer's eyes from bright ambient light. In some cases, a wearer chooses to use a tinted shield to protect their eyes from bright ambient light. In other instances, a wearer chooses to use tinted eyeglasses. In either case, during times when ambient light is minimal, these solutions generally require that a wearer change from the tinted face shield and/or tinted eyeglasses to a non-tinted face shield and/or non-tinted eyeglasses. Aside from the cost of separate tinted and non-tinted face shields and/or eyeglasses, the need to switch between face shields and or eyeglasses is inconvenient and undesirable.

Like helmets designed specifically for use in automotive applications, motorcycle helmets must be capable of withstanding a practical level of impact and compressive forces. Due to the respective environments that a motorcycle helmet is subjected and that to which an automotive-type helmet is subjected, the specific types of impact and compressive load requirements are sometimes different for motorcycle helmets (i.e., helmets designed with the intent for use in motorcycle applications) and automobile helmets (i.e., helmets designed with the intent for use in automobile and similar race vehicle applications). However, aside from the specific load requirements, both types of helmets have a key requirement of protecting a wearer's head in the case of an accident.

Motorcycle helmets have the additional requirement of needing to offer increased visibility with respect to a helmet designed specifically for automotive application. For example, a helmet designed specifically for use in motorcycle applications preferably provides for a larger field of view. The reason for such larger field of view is that a motorcyclist must be able to readily view overhead stoplights and readily view the road surface immediately in front of his or her motorcycle. To this end, motorcycle helmets generally have a wider and taller eye port than does an automobile helmets. The wider and taller eye port provides reduced the need for a motorcyclist to move his or her head to see objects outside of their immediate line of sight (e.g., objects within their normal peripheral view).

By very nature, a motorcyclist has safety issues other than does a wearer of a helmet in an automobile application. One such issue that is of paramount importance to a motorcyclist is conspicuity (i.e., other motorists recognizing the presence of a motorcyclist). A motorcyclist not being recognized by a nearby motorist is one of the greatest threats to the safety of the motorcyclist. The sooner and more easily the motorcyclist can be seen by nearby motorists, the safer the motorcyclist will be.

Therefore, a face shield for a motorcycle helmet that enhances visibility and/or conspicuity is useful.

### SUMMARY OF THE DISCLOSURE

Embodiments of the present invention eliminate the need for a wearer of a helmet to switch between a tinted face shield and/or eyewear and a non-tinted face shield and/or eyewear. This reduces the inconvenience and expense associated with switching between such face shields and/or eyewear. Furthermore, embodiments of the present invention enhance visibility of critical roadway conditions while exhibiting tinted characteristics to protect a wearer's eyes from bright ambient light. Accordingly, the present invention advantageously overcomes one or more shortcomings associated with conventional face shields for helmets.

In one embodiment of the present invention, a face shield for a helmet comprises a face shield body including end portions and a central portion extending between the end portions. Each one of the end portions is configured for enabling attachment of the face shield to a respective attachment portion of a helmet. The central portion is made from transparent polymeric material. The polymeric material in a first area of the central portion exhibits non-photochromic properties at least one layer of the polymeric material in a second area of the central portion exhibits photochromic properties.

In another embodiment of the present invention, a helmet assembly comprises a helmet body and a face shield body. The helmet body has an eyeport extending therethough. The face shield body includes end portions and a central portion extending between the end portions. The end portions are each movably attached to respective portions of the helmet body in a manner enabling the face shield to be moved between an open position and a closed position. The central portion covers the eyeport when the face shield is in the closed position. The central portion is made from transparent polymeric material. The polymeric material in a first area of the central portion exhibits non-photochromic properties. One or more layers of the polymeric material in a second area of the central portion exhibit photochromic properties. At least a portion of the first area and at least a portion of the second area cover respective areas of the eyeport when the face shield is in the closed position.

In another embodiment of the present invention, a method for making a face shield for a motorcycle is provided. The method includes providing a face shield body including end portions and a central portion extending between the end portions. Each one of the end portions is configured for enabling attachment of the face shield body to a respective attachment portion of a helmet and the central portion is made from transparent polymeric material. After providing the face shield body, a mask is formed over at least a first area of the central portion of the face shield body such that at least one surface of the central portion in a second area of the central portion is non-masked. Next, a layer of photochromic polymeric material is formed on the non-masked second area of the central portion. Thereafter, the mask is removed.

Turning now to specific aspects of the present invention, in at least one embodiment, the second area of the central portion has a width less than an overall width of the central portion.

In at least one embodiment of the present invention, the material of the central portion in the first area consists of a single layer of polymeric material exhibiting non-photochromic properties, the material of the central portion in the second area consists of a plurality of layers of material, a first one of the layers in the second area consists of polymeric material exhibiting non-photochromic properties and a second one of the layers in the second area consists of polymeric material exhibiting photochromic properties.

In at least one embodiment of the present invention, the material of the second area consists of a single layer of material.

In at least one embodiment of the present invention, the first area is partially bound by a lower edge of the face shield body and by the second area.

In at least one embodiment of the present invention, the first area is partially bound by the end portions of the face shield body.

In at least one embodiment of the present invention, the second area is partially bound by an upper edge of the face shield body.

In at least one embodiment of the present invention, the second area is partially bound by the lower edge of the face shield body.

In at least one embodiment of the present invention, the central portion includes a third area, a highly reflective material is provided on the face shield body in the entire third area, the second area is bound entirely between the first area, by the third area and by a lower edge of the face shield body, and the first area is entirely bound by a lower edge of the face shield body of the face shield body and by the second area.

In at least one embodiment of the present invention, the first area of the central portion covers at least a portion of a lower edge of the eyeport of a helmet.

In at least one embodiment of the present invention, the first area of the central portion covers a first area of the eyeport when the face shield body is in the closed position, the second area of the central portion covers a second area of the eyeport when the face shield body is in the closed position and the first area of the eyeport and the second area of the eyeport jointly comprise an entire area of the eyeport.

In at least one embodiment of the present invention, the first area of the eyeport is partially bound by a lower edge of the eyeport.

In at least one embodiment of the present invention, forming the mask includes forming the mask on an interior surface and an exterior surface of the face shield body.

In at least one embodiment of the present invention, forming the mask includes forming the mask on the interior surface and the exterior surface of face shield body in the first area, and forming the mask on the exterior surface of the face shield body in the second area.

In at least one embodiment of the present invention, forming the layer of photochromic polymeric material includes providing a liquefied layer of photochromic material on the non-masked second area of the central portion and applying heat within a prescribed temperature range to the liquefied layer of photochromic material.

These and other objects, embodiments advantages and/or distinctions of the present invention will become readily apparent upon further review of the following specification, associated drawings and appended claims.

### BRIEF DESCRIPTION OF THE DRAWINGS

FIG. 1 depicts an embodiment of a face shield in accordance with the present invention, wherein the face shield is in a first sensichromic state.

FIG. 2 depicts the face shield depicted in FIG. 1 in a second sensichromic state.

FIG. 3 is a cross-sectional view taken along the line 3-3 in FIG. 2.

FIG. 4 depicts an alternate embodiment of the face shield depicted 1, wherein a highly reflective coating is provided on the face shield.

### DETAILED DESCRIPTION OF THE DRAWING FIGURES

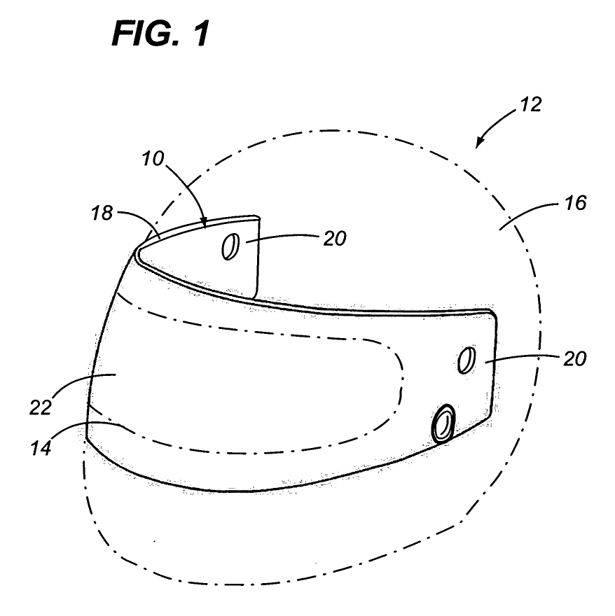

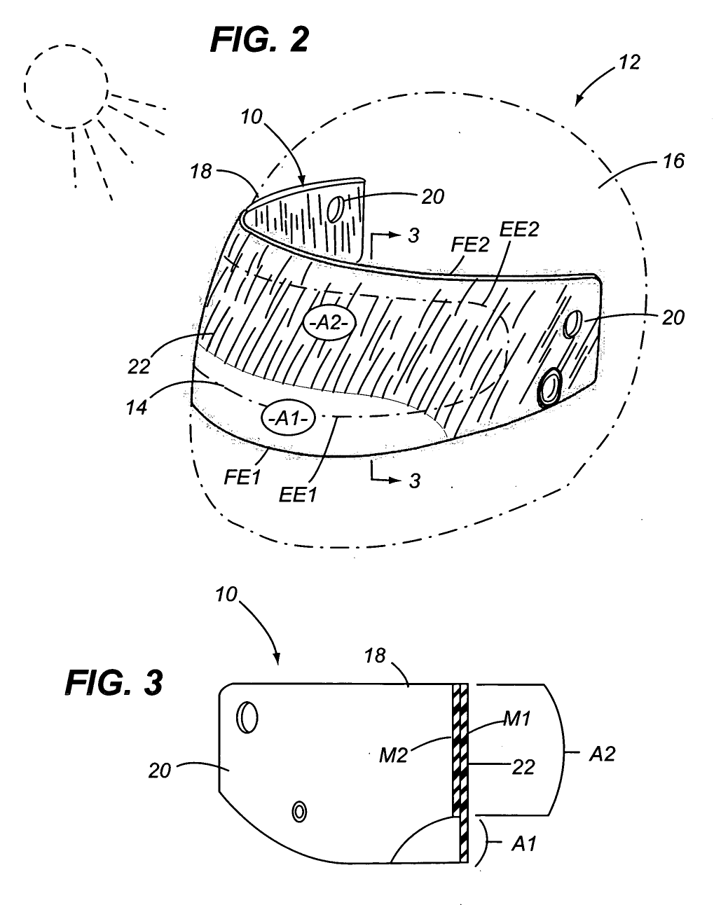

FIGS. 1 and 2 depict an embodiment of a face shield in accordance with the present invention, which is referred to herein as the face shield 10. The face shield 10 is mounted on a helmet 12, which includes an eyeport 14 extending through a helmet body 16 of the helmet 12. Accordingly, the helmet 12 is an embodiment of a helmet in accordance with the present invention.

The face shield 10 includes a face shield body 18. The face shield body 18 includes end portions 20 and a central portion 22 extending between the end portions 20. The end portions 20 are each movably attached to respective portions of a helmet body 16 of the helmet 12. Such attachment in a movable manner enables the face shield 10 to be moved between a closed position (shown) and an open position (not shown). For example, each one of the end portions 20 may be pivotally attached to a respective portion of the helmet body 16 thereby enabling the face shield 10 to be pivoted between the closed position and the open position. An example of the open position is a conventional open position where the face shield 10 is pivoted to a position at least partially above the eyeport 14. It is disclosed herein that a face shield in accordance with the present invention is not limited by a particular mounting configuration or configuration of the end portions 20.

The central portion 22 of the face shield body 18 fully covers the eyeport 14 when the face shield 10 is in the closed position. As is the case with most conventional helmets, the face shield body 18 is in very close proximity to the eyeport when the face shield 10 is in the closed position. Preferably, but not necessarily, a sealing member (not shown) extends around the perimeter of the eyeport 14 and is engaged by the face shield body 18 when the face shield 10 is in the closed position.

Due to such close proximity of the face shield body 18 to the eyeport 14 when the face shield 10 is in the closed position, a total viewable area of a wearer of the helmet 12 for a given head position is generally limited by a total area of the eyeport 14 rather than by the overall area of the central portion 22 of the face shield body 18. Accordingly, an upper part of the central portion 22 (e.g., the part of the central portion that resides above the eyeport 14 when the face shield 10 is in the closed position) is not useful for being viewed through when the face shield 10 is in the closed or open positions. Similarly, a lower part of the central portion 22 (e.g., the part of the central portion that resides above the eyeport 14 when the face shield 10 is in the closed position) is not useful for being viewed through when the face shield 10 is in the closed position.

The central portion 22 of the face shield body 18 is made from transparent polymeric material. Preferably, but not necessarily, the end portions 20 are integrally formed with the central portion 22 and are, thus, made from the same transparent polymeric material. Polycarbonate is a preferred example of a transparent polymeric material from which the end portions 20 and central portion 22 are made.

In accordance with the present invention, various different areas of the central portion 22 have respective photochromic properties. Photochromic properties are defined herein to include a change in a degree of light transmission (i.e., light transmissivity) through a material being dependent on a respective amount of light intensity (e.g., UV light content). For example, a material exhibiting photochromic properties transmits a first degree of a given amount of light when exposed to a first amount of sunlight and transmits a second degree of a given amount of light when exposed to a second amount of sunlight. In this manner, light transmission of at least a portion of a face shield in accordance with the present invention is dependent upon an amount of direct and/or reflected sunlight that impinges upon the face shield. A color change is generally noted in a material exhibiting photochromic properties. Non-photochromic properties are defined herein to include a degree of light transmission through a material being substantially independent of a respective amount of light intensity (e.g., UV light content) that impinges upon the material. Accordingly, a face shield or portion thereof that exhibits non-photochromic properties does not change color and/or does not have a variable light transmissivity.

As depicted in FIG. 1, an entire area of the central portion 22 of the face shield body 18 exhibits substantially full transparency (e.g., is clear or is at a base-level of transmissivity) when exposed to a first degree of light intensity (e.g., a night time level of sunlight). In this manner, the face shield body 18 provides a first level of light transmissivity (e.g., a maximum level of light intensity) during exposure to the first degree of light intensity. The first level of light transmissivity is a relatively high level of light transmissivity thus providing for preferred visibility through the face shield body 18 during exposure to the first degree of light intensity.

As depicted in FIG. 2, when exposed to a second degree of light intensity (e.g., a bright sunlight) which is greater than the first degree of light intensity, a first area A1 of the central portion 22 of the face shield body 18 exhibits substantially full transparency (e.g., is clear or is at the base-level of transmissivity) and a second area A2 of the central portion 22 of the face shield body 18 exhibits substantially reduced transparency (e.g., a second level of light transmissivity). The second area A2 of the central portion 22 exhibits photochromic property, thus enabling the change in level of transmissivity dependent upon light intensity. In this manner, the first area A1 of the central portion 22 continues to provide the first level of light transmissivity during exposure to the second degree of light intensity while the second area A2 of the central portion 22 provides the reduced level of transmissivity. The second level of light transmissivity is a relatively low level of light transmissivity thus providing for preferred visibility through the face shield body 18 during the second area A2 of the central portion 22 during exposure to the second degree of light intensity. As depicted in FIG. 2, the material constituting the central portion 22 in the first area A1 exhibits non-photochromic properties whereas the material constituting the central portion 22 in the second area A2 exhibits photochromic properties.

Advantageously, in comparison to a conventional statically tinted face shield having a central portion that exhibits a relatively low level of light transmissivity, the first area A1 of the central portion 22 provides for preferred visibility of roadway surfaces (i.e., visibility that is not reduced or impaired by reduced light transmissivity). However, the second area A2 of the central portion 22 provides for similar protection from bright sunlight as is provided for by a conventional statically tinted face shield. The present invention is not limiting as to the size, shape and/or position of the first area A1 of the central portion 22 relative to the second area A2 of the central portion 22. This, the specific size, shape and/or position of the first area A1 of the central portion 22 relative to that of the second area A2 of the central portion 22 is configurable during design/manufacture of a face field in accordance with the present invention to provide a desired result.

The first area A1 of the central portion 22 is entirely bound between a lower edge FE1 of the face shield body and the second area A2. The second area A2 of the central portion 22 is bound between the first area A1 of the central portion 22, the lower edge FE1 of the face shield body 18, the end portions 20 and an upper edge FE2 of the face shield body 18. Optionally, the first are A1 of the central portion may be wider such that it fully bisects the second area A2 of the central portion 22 and the lower edge FE1 of the face shield body 18.

As depicted in FIG. 2, when the face shield 10 is in the closed position, a portion of the first area A1 of the central portion 22 covers a first area of the eyeport 14 and a portion of the second area A2 of the central portion 22 covers a second area of the eyeport 14. The first and second areas of the eyeport 14 jointly define an entire area of the eyeport 14. Additionally, the first area A1 of the central portion 22 covers a portion of a lower edge EE1 of the eyeport 14 and the second area A2 of the central portion 22 covers a portion of an upper edge EE2 of the eyeport 14. As will be understood from the preceding description, the first area of the eyeport 14 is partially bound by the lower edge EE1 of the eyeport 14 and the second area of the eyeport 14 is partially bound by the upper edge EE2 of the eyeport 14.

Referring now to FIGS. 2 and 3, first area A1 of the central portion 22 consists of a single layer of polymeric material exhibiting non-photochromic properties and the second area A2 of the central portion 22 consists of a plurality of layers of material. The single layer of polymeric material in the first area A1 and a first layer M1 of material in the second area A2 consists of polymeric material exhibiting non-photochromic properties and are jointly constituted by the a base structure (i.e., a base layer) of the face shield body 18. A second layer M2 of material in the second area A2 consists polymeric material exhibiting photochromic properties. Preferably, but not necessarily, the second layer M2 of material is provided on an interior surface of the base structure. In one preferred embodiment, the base structure is a commercially available and/or known face shield, which is made from clear or tinted plastic.

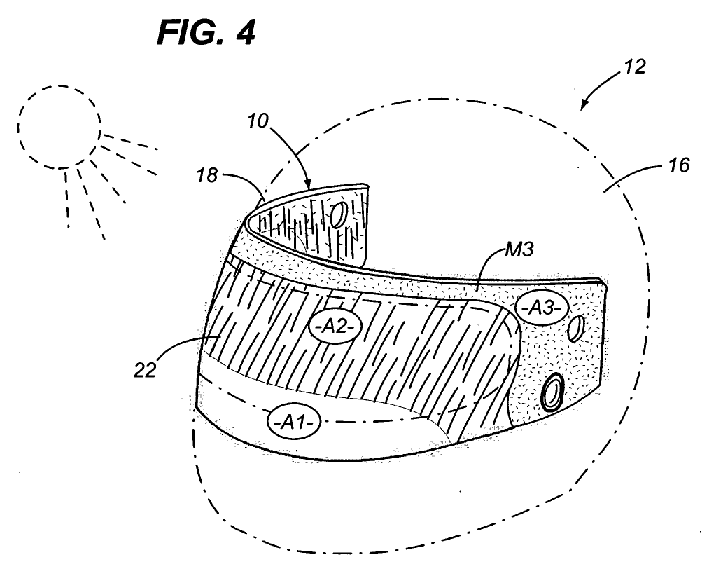

To further enhance safety, it is disclosed herein that the face shield 10 depicted in FIGS. 1-3 may include a layer of a highly reflective material M3 formed thereon. As depicted in FIG. 4, the face shield body 22 includes a third area A3 that encompasses a portion of the central portion 22 of the face shield body 18 and, optionally all of the end portions 20. The highly reflective material M3 is provided on the face shield body in the third area A3. The second area A2 is bound entirely between the first area A1, the third area A3 and the lower edge FE1 of the face shield body 18. Examples of the highly reflective material M3 included material providing a mirror finish (e.g., a chrome-like finish) and material providing a reflective finish (e.g., a 3M diamond grade or high-intensity grade reflective material or the like).

Turning now to a discussion of a preferred method for making a face shield in accordance with the present invention, it is important for a reader to understand known approaches for providing a base structure (e.g., a plastic sheet) with photochromic properties. Known approaches for providing a base structure with photochromic properties include, but are not limited to, including a photochromic reagent in a polymeric resin from which the base structure is made, coating a pre-formed base structure with a layer of material that exhibits photochromic properties, applying a preformed layer of material that exhibits photochromic properties onto a pre-formed base structure and co-molding/overmolding a material exhibiting photochromic properties with a material exhibiting non-photochromic properties. U.S. Pat. Nos. 5,531,940; 5,975,696; and 6,773,108 disclose respective approaches for coating a pre-formed base structure with a layer of material that exhibits photochromic properties in a manner suitable for enabling a face shield in accordance with the present invention to be fabricated. U.S. Pat. No. 6,367,930 discloses an approach for co-molding/overmolding a material exhibiting photochromic properties with a material exhibiting non-photochromic properties in a manner suitable for enabling a face shield in accordance with the present invention to be fabricated. U.S. Pat. Nos. 5,411,835 and 6,228,499, jointly, and U.S. Pat. No. 6,065,836 disclose respective methods for applying a film exhibiting photochromic properties in a manner suitable for enabling a face shield in accordance with the present invention to be fabricated. Each of the patents disclosed above is incorporated herein in its entirety by reference.

In a preferred method for fabricating a face shield in accordance with the present invention, a layer of photochromic material is formed on a preformed face shield (i.e., a pre-formed base structure). The method includes providing a face shield body including end portions and a central portion extending between the end portions. A mask is then formed over at least a first area of the central portion (e.g., the first area A1 discussed above) of the face shield such that at least one surface of the central portion in a second area of the central portion (e.g., the second area A2 discussed above) is non-masked. Preferably, but not necessarily, forming the mask includes forming the mask on an interior surface and an exterior surface of the face shield body in the first area and forming the mask on the exterior surface of the face shield body in the second area. Examples of methods for forming the mask include applying, adhering and/or bonding a preformed mask (e.g., solid or semi-solid composition) and applying, drying and/or curing a liquefied mask material. In some instances, applying, adhering, bonding, drying and/or curing include applying heat within a prescribed temperature range. After forming the mask, a layer of photochromic polymeric material is formed on the non-masked second area of the central portion, followed by the mask being removed.

Similarly, the highly reflective material M3 depicted in FIG. 3 may be formed through the use of masks. For example, masking of the first area A2 and the second area A3 will enable a layer of the highly reflective material to be formed in the non-masked area of the face shield. Such layer may be formed by any number of operations such as, but not limited to, immersion coating, spraying, transfer deposition, and ionic attraction. Such operations may require one or more other known operation for providing suitable surface preparation and/or final adhesion/bonding.

It is disclosed herein that light-sensitive transmissivity is provided by virtue of a material exhibiting photochromic properties. It is also disclosed herein that such light-sensitive transmissivity may also be provided via a material exhibiting electrochromic properties. Electrochromic properties are defined herein to include a change in a degree of light transmission (i.e., light transmissivity) through a material being dependent on a respective amount of applied voltage and/or current. (e.g., as supplied from a photocell and/or battery). Accordingly, sensichromic properties are defined herein to be photochromic properties and/or electrochromic properties.

In the preceding detailed description, reference has been made to the accompanying drawings that form a part hereof, and in which are shown by way of illustration specific embodiments in which the present invention may be practiced. These embodiments, and certain variants thereof, have been described in sufficient detail to enable those skilled in the art to practice embodiments of the present invention. It is to be understood that other suitable embodiments may be utilized and that logical, mechanical, chemical and electrical changes may be made without departing from the spirit or scope of such inventive disclosures. To avoid unnecessary detail, the description omits certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. A face shield for a helmet, comprising:
a face shield body including end portions and a central portion extending between said end portions, wherein each one of said end portions is configured for enabling attachment of the face shield to a respective attachment portion of a helmet, wherein the central portion is made from transparent polymeric material, wherein said polymeric material in a first area of the central portion exhibits non-sensichromic properties and wherein at least one layer of said polymeric material in a second area of the central portion exhibits sensichromic properties.

2. The face shield of claim 1 wherein the second area has a width less than an overall width-of the central portion.

3. The face shield of claim 1 wherein:
said material of the central portion in the first area consists of a single layer of polymeric material exhibiting non-sensichromic properties;
said material of the central portion in the second area consists of a plurality of layers of material;
a first one of said layers in the second area consists of polymeric material exhibiting non-sensichromic properties; and
a second one of said layers in the second area consists of polymeric material exhibiting sensichromic properties.

4. The face shield of claim 1 wherein said material of the second area consists of a single layer of material.

5. The face shield of claim 1 wherein the first area is partially bound by a lower edge of the central portion of the face shield body and by the second area.

6. The face shield of claim 5 wherein the first area is partially bound by the end portions of the face shield body.

7. The face shield of claim 5 wherein the second area is partially bound by an upper edge of the central portion.

8. The face shield of claim 7 wherein the second area is partially bound by the lower edge of the central portion.

9. The face shield of claim 1 wherein:
the central portion includes a third area;
a highly reflective material is provided on the face shield body in the entire third area;
the second area is bound entirely between the first area, the third area and a lower edge of the central portion; and
the first area is entirely bound by a lower edge of the central portion of the face shield body and by the second area.

10. The face shield of claim 1 wherein:
the second area has a width less than an overall width of the central portion;
said material of the central portion in the first area consists of a single layer of polymeric material exhibiting non-sensichromic properties;
said material of the central portion in the second area consists of a plurality of layers of material;
a first one of said layers in the second area consists of polymeric material exhibiting non-sensichromic properties;
a second one of said layers in the second area consists of polymeric material exhibiting sensichromic properties;
the first area is partially bound by a lower edge of the central portion of the face shield body and by the second area;
the second area is partially bound by an upper edge of the central portion; and
the second area is partially bound by the lower edge of the central portion.

11. A helmet assembly, comprising:
a helmet body having an eyeport extending therethough; and
a face shield body including end portions and a central portion extending between said end portions, wherein said end portions are each movably attached to respective portions of the helmet body in a manner enabling the face shield body to be moved between an open position and a closed position, wherein the central portion covers the eyeport when the face shield body is in the closed position, wherein the central portion is made from transparent polymeric material, wherein said polymeric material in a first area of the central portion exhibits non-sensichromic properties, wherein at least one layer of said polymeric material in a second area of the central portion exhibits sensichromic properties and wherein at least a portion of the first area and at least a portion of the second area cover respective areas of the eyeport when the face shield body is in the closed position.

12. The helmet assembly of claim 11 wherein the first area of the central portion covers at least a portion of a lower edge of the eyeport.

13. The helmet assembly of claim 11 wherein:
the first area of the central portion covers a first area of the eyeport when the face shield body is in the closed position;
the second area of the central portion covers a second area of the eyeport when the face shield body is in the closed position; and
the first area of the eyeport and the second area of the eyeport jointly comprise an entire area of the eyeport.

14. The helmet assembly of claim 13 wherein the first area of the eyeport is partially bound by a lower edge of the eyeport.

15. A method for making a face shield for a motorcycle, comprising:
providing a face shield body including end portions and a central portion extending between said end portions, wherein each one of said end portions is configured for enabling attachment of the face shield body to a respective attachment portion of a helmet and wherein the central portion is made from transparent polymeric material,
forming a mask over at least a first area of the central portion of the face shield body such that at least one surface of the central portion in a second area of the central portion is non-masked;
forming a layer of sensichromic polymeric material on the non-masked second area of the central portion; and
removing the mask.

16. The method of claim 15 wherein forming the mask includes forming the mask on an interior surface and an exterior surface of the face shield body.

17. The method of claim 16 wherein forming the mask includes:
forming the mask on the interior surface and the exterior surface of face shield body in the first area; and
forming the mask on the exterior surface of the face shield body in the second area.

18. The method of claim 15 wherein:
forming the mask includes forming the mask on the interior surface and the exterior surface of face shield body in the first area;
the first area is bound by a lower edge of the central portion of the face shield body and by the second area;
the second area is partially bound by an upper edge of the central portion; and
the second area is partially bound by the lower edge of the central portion.

19. The method of claim 15 wherein forming the layer of sensichromic polymeric material includes:
providing a liquefied layer of sensichromic material on the non-masked second area of the central portion; and
applying heat within a prescribed temperature range to the liquefied layer of sensichromic material.

20. The method of claim 19 wherein:
forming the mask includes forming the mask on the interior surface and the exterior surface of face shield body in the first area;
the first area is bound by a lower edge of the central portion of the face shield body and by the second area;
the second area is partially bound by an upper edge of the central portion; and
the second area is partially bound by the lower edge of the central portion.
